(19) United States
(12) Patent Application Publication
(10) Pub. No.: US 2014/0148470 A1
(43) Pub. Date: May 29, 2014

(54) PYRIMIDINE COMPOUNDS FOR TREATING HAIRLOSS

(71) Applicants: Andreas SCHNAPP, Biberach an der Riss (DE); Jeffrey Adam ENCINAS, Ashiya (JP); Andy FOWLER, Guildford (GB); Takeshi KONO, Osaka (JP); Katsuhiro UTO, Higashinada (JP)

(72) Inventors: Andreas SCHNAPP, Biberach an der Riss (DE); Jeffrey Adam ENCINAS, Ashiya (JP); Andy FOWLER, Guildford (GB); Takeshi KONO, Osaka (JP); Katsuhiro UTO, Higashinada (JP)

(73) Assignee: Boehringer Ingelheim International GmbH, Ingelheim am Rhein (DE)

(21) Appl. No.: 14/082,256

(22) Filed: Nov. 18, 2013

(30) Foreign Application Priority Data
Nov. 23, 2012 (EP) 12194123.1

(51) Int. Cl. A61K 8/49; A61Q 7/00

(52) U.S. Cl. CPC A61K 8/4953; A61Q 7/00
USPC 514/256

(57) ABSTRACT

The present invention relates to compounds of formula (I) or pharmaceutically acceptable salts thereof for use in the treatment or prevention of hairloss,

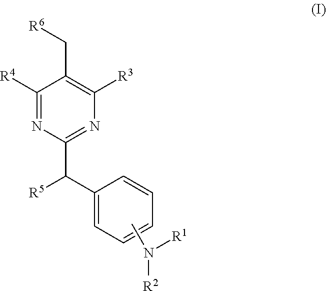

wherein R¹, R², R³, R⁴, R⁵ and R⁶ have one of the meanings as indicated in the specification and claims, to pharmaceutical compositions containing said compounds or pharmaceutically acceptable salts thereof, to the use of said compounds or pharmaceutically acceptable salts thereof for the manufacture of a medicament useful for the treatment or prevention of hairloss, to a method of treating or preventing hairloss as well as to a method of stimulating hair growth.

The present invention relates to compounds of formula (I) or pharmaceutically acceptable salts thereof for use in the treatment or prevention of hairloss,

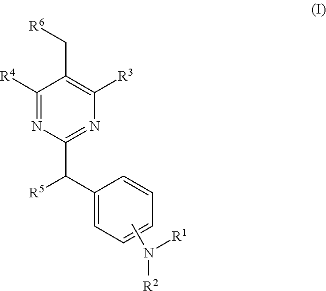

wherein R¹, R², R³, R⁴, R⁵ and R⁶ have one of the meanings as indicated in the specification and claims, to pharmaceutical compositions containing said compounds or pharmaceutically acceptable salts thereof, to the use of said compounds or pharmaceutically acceptable salts thereof for the manufacture of a medicament useful for the treatment or prevention of hairloss, to a method of treating or preventing hairloss as well as to a method of stimulating hair growth.

### BACKGROUND OF THE INVENTION

Hair loss has a negative impact on the self-respect of both women and men. The most common type of hairloss effecting both women and men is androgenic alopecia (AGA). In men AGA is characterized by pattern hairloss with bitemporal recession and vertex baldness (MPHL). In women AGA produces female pattern hair loss (FPHL) with a diffuse hairloss over the mid-frontal scalp.

In both forms the hairloss occurs as a result of speckeled hair follicle miniaturization within follicular units and is characterized of hair cycles with a shortened growth (anagen) phase.

In man testosterone is an essential factor involved in the development of AGA. In addition, inflammatory and genetic components have been hypothezised to play a function, too. In women, the impact of androgens on FPHL is unclear. Hair follicle miniaturization and a shortened anagen phase is most likely also the cause of other forms of hair loss.

Treatment options to arrest hair loss progression and stimulate partial hair regrowth include androgen receptor antagonists (spironolactone and cyproterone acetate), the 5α-reductase inhibitor, finasteride, and the androgen-independent hair growth stimulator, minoxidil.

The CRTH2 (chemoattractant receptor-homologous molecule expressed on Th2 cells) protein, also known as GPR44, is a G-protein coupled receptor (GPCR) which is amongst other ligands most strongly activated by prostaglandin D2 (PGD2). PGD2 is a product of prostaglandin D2 synthase (PTGDS).

Garza et al (Science TransMed, 2012, 4,(126):126ra34), showed that PTGDS is upregulated both on the mRNA and protein levels in bald scalp of men with AGA and that PGD2 levels are higher in bald scalp compared to normal scalp, too. Furthermore, they showed that PTGDS protein and PGD2 levels increase before the regression phase of human hair follicles. Administration of PGD2 to explanted human hair follicles inhibited hair growth. When applied topically to the skin of wild-type mice, PGD2 inhibited hair growth, too, but not when applied to CRTH2 (GPR44) knockout mice, suggesting that indeed CRTH2 is the responsible receptor.

Therefore, agents that antagonize the effects of PGD2 at the CRTH2 receptor should be useful for the treatment of AGA, and other forms of hairloss related to enhanced CRTH2 activity.

WO 2007/149312 A2 relates to the use of compounds capable of decreasing the PDG2 level or activity, a downstream signaling or receptor pathway thereof, or PGD2 synthase level or activity, such as CRTH2 antagonists, in methods of treating androgenic alopecia.

It is an objective of the present invention to provide compounds for the treatment of AGA and other conditions of hairloss having enhanced CRTH2 activity or an enhanced PGD2-CRTH2 axis. Said compounds should allow for treating AGA and other forms of hairloss related to enhanced CRTH2 activity.

According to the present invention this objective is achieved by the compounds of formula (I) as defined herein and previously disclosed in WO 2004/096777 A1.

### DETAILED DESCRIPTION OF THE INVENTION

The present invention relates to compounds of formula (I) or pharmaceutically acceptable salts thereof for use in the treatment or prevention of hairloss, in particular hairloss in humans,

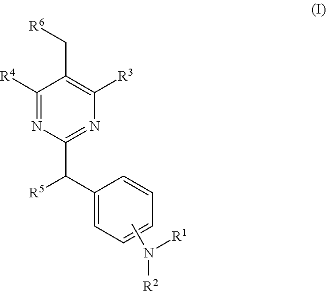

wherein R¹ represents hydrogen,

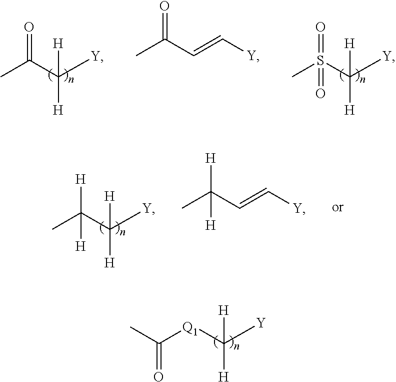

in which
n represents an integer of 0 to 6;
Q₁ represents—NH—, —N(C₁₋₆ alkyl)-, or —O—;
Y represents hydrogen, C₁₋₆ alkyl, C₃₋₈, cycloalkyl optionally substituted by C₁₋₆ alkyl, C₃₋₈ cycloalkyl fused by benzene, aryl or heteroaryl, wherein said aryl and heteroaryl are optionally substituted at a substitutable position with one or more substituents selected from the group consisting of cyano, halogen, nitro, guanidino, pyrrolyl, sulfamoyl, C₁₋₆ alkylaminosulfonyl, di(C₁₋₆ alkyl) aminosulfonyl, phenyloxy, phenyl, amino, C₁₋₆ alkylamino, di(C₁₋₆)alkylamino, C₁₋₆ alkoxycarbonyl, C₁₋₆ alkanoyl, C₁₋₆ alkanoylamino, carbamoyl, C₁₋₆ alkylcarbamoyl, di(C₁₋₆ alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen, or aryl fused by 1,3-dioxolane;
R² represents hydrogen or C₁₋₆ alkyl;
R³ represents halogen, C₁₋₆ alkoxy optionally mono-, di-, or tri-substituted by halogen,

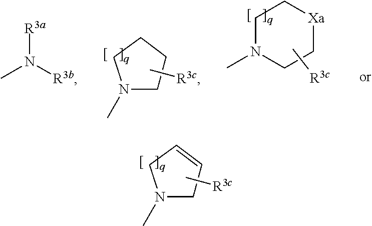

in which
R^{3a} and R^{3b} independently represent C₃₋₈, cycloalkyl, or C₁₋₆ alkyl, which C₁₋₆ alkyl is optionally substituted by hydroxy, carboxy, C₃₋₈, cycloalkyl, carbamoyl, C₁₋₆ alkylcarbamoyl, aryl-substituted C₁₋₆ alkylcarbamoyl, C₁₋₆ alkylcarbamoyl, di (C₁₋₆ alkyl) carbamoyl, C₃₋₈, cycloalkylcarbamoyl, C₃₋₈ heterocyclocarbonyl, (C₁₋₆)alkylamino, di(C₁₋₆) alkylamino or C₁₋₆ alkoxy,
q represents an integer of 1 to 3;
R^{3c} represents hydrogen, hydroxy, carboxy, or C₁₋₆ alkyl optionally substituted by hydroxy, carboxy or (phenyl-substituted C₁₋₆ alkyl) carbamoyl;
Xa represents —O—, —S— or —N(R^{3d})—, in which R^{3d} represents C₁₋₆ alkyl;
R⁴ represents hydrogen, halogen, C₁₋₆ alkoxy, di(C₁₋₆ alkyl)amino or C₁₋₆ alkyl optionally substituted by C₁₋₆ alkoxy, or mono-, di-, or tri-substituted by halogen;
R⁵ represents hydrogen, or C₁₋₆ alkyl; and
R⁶ represents carboxy, carboxamide, nitrile or tetrazolyl.

The compounds of formula (I) according to the present invention are particularly suitable for treating androgenic alopecia and other forms of hairloss related to enhanced CRTH2 activity.

Accordingly the present invention further relates to pharmaceutical compositions for use in the prevention or treatment of hair loss, in particular hair loss in humans, containing at least one compound of formula (I) or a pharmaceutically acceptable salt thereof as defined herein.

The present invention further relates to a method of treating or preventing hair loss, in particular hair loss in a human, said method comprising the step of administering a therapeutically effective amount of at least one compound of formula (I) or a pharmaceutically acceptable salt thereof as defined herein.

The present invention further relates to a method of stimulating hair growth, in particular hair growth in a human, said method comprising the step of administering a therapeutically effective amount of at least one compound of formula (I) or a pharmaceutically acceptable salt thereof as defined herein.

The present invention further relates to the use of at least one compound of formula (I) or a pharmaceutically acceptable salt thereof as defined herein for the manufacture of a medicament useful for the treatment or prevention of hairloss, in particular hairloss in human.

The present invention further relates to the use of at least one compound of formula (I) or a pharmaceutically acceptable salt thereof as defined herein for the manufacture of a medicament useful for stimulating hair growth, in particular hair growth in a human.

The activity in a whole cell eosinophil shape change assay of the compounds of the invention can be determined, for example, according to the following references: (i) Mathiesen J M, Ulven T, Martini L, Gerlach L O, Heinemann A, Kostenis E. Identification of indol derivatives exclusively interfering with a G protein-independent signalling pathway of the prostaglandin D2 receptor CRTH2. Mol. Pharmacol. 2005 August; 68(2):393-402; (ii) Schuligoi R, Schmidt R, Geisslinger G, Kollroser M, Peskar B A, Heinemann A. PGD2 metabolism in plasma: kinetics and relationship with bioactivity on DP1 and CRTH2 receptors. Biochem Pharmacol. 2007 Jun. 30; 74(1):107-17; (iii) Royer J F, Schratl P, Carrillo J J, Jupp R, Barker J, Weyman-Jones C, Beri R, Sargent C, Schmidt J A, Lang-Loidolt D, Heinemann A. A novel antagonist of prostaglandin D2 blocks the locomotion of eosinophils and basophils. Eur J Clin Invest. 2008 September; 38(9):663-71.

The chemical stability of the compounds of the invention can be determined, for example, under the following conditions: (i) 3 days incubation at 60° C. in 0.1 N HCl (hydrolytic stability under acidic conditions); (ii) 3 days incubation at 60° C. in pH 4.0 buffer solution (hydrolytic stability under weakly acidic conditions); (iii) 3 days incubation at 60° C. in pH 7.4 buffer solution (hydrolytic stability at physiological pH); (iv) 3 days incubation at 20° C. in 0.3% hydrogen peroxide (stability against oxidants); (v) 24 h incubation under UV-radiation (lambda=300-800 nm, P=250 W/m2) in water (stability against light). The kinetics of degradation can, for example, be determined by HPLC analysis.

The pharmacokinetic properties (PK) of the compounds of the invention can be determined in pre-clinical animal species, for example, mouse, rat, dog, guinea pig, mini pig, cynomolgus monkey, rhesus monkey. The pharmacokinetic properties of a compound can be described, for example, by the following parameters: Mean residence time, half-life, volume-of-distribution, AUC (area under the curve), clearance, bioavailability after oral administration.

### USED TERMS AND DEFINITIONS

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals or moieties defined below, the number of carbon atoms is often specified preceding the group. As an example “C₁-C₆-alkyl” means an alkyl group or radical having 1 to 6 carbon atoms.

In general, for groups comprising two or more subgroups, the last named group is the radical attachment point.

Unless otherwise specified, conventional definitions of terms control and conventional stable atom valences are presumed and achieved in all formulas and groups.

In general, all tautomeric forms and isomeric forms and mixtures, whether individual geometric isomers or optical isomers or racemic or non-racemic mixtures of isomers, of a chemical structure or compound are comprised, unless the specific stereochemistry or isomeric form is specifically indicated in the compound name or structure.

The term “substituted” as used herein, means that any one or more hydrogens on the designated atom, moiety or radical is replaced with a selection from the indicated group of radicals, provided that the designated atom's normal valence is not exceeded, and that the substitution results in a stable compound.

The compounds disclosed herein can exist as pharmaceutically acceptable salts. The present invention includes compounds in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCH, Zurich, Switzerland, 2002).

The term “pharmaceutically acceptable salt,” as used herein, represents salts or zwitterionic forms of the compounds disclosed herein which are water or oil-soluble or dispersible and pharmaceutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphor sulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylene sulfonate, methane sulfonate, naphthylene sulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric acid, hydrobromic acid, sulfuric acid and phosphoric acid, and organic acids such as oxalic acid, maleic acid, succinic acid and citric acid. Salts can also be formed by coordination of the compounds with an alkali metal or alkaline earth ion. Hence, the present invention comprises sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine and piperazine.

While it may be possible for the compounds of the present invention to be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carrier and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers and excipients may be used as suitable and as understood in the art; e.g. in Remington's Pharmaceutical Sciences. The pharmaceutical compositions disclosed herein may be manufactured in any manner known in the art, e.g., by means of conventional mixing, dissolving, granulating, dragee-making, levigating, emulsifying, encapsulating, entrapping or compression processes.

The term “alkyl” as used herein and for the alkyl moieties of alkoxy, alkanoyl, alkylcarbamoyl, alkylthio, alkylamino, alkylaminocarbonyl, alkylaminosulphonyl, alkylsulphonylamino, alkoxycarbonyl, alkoxycarbonylamino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

The term “alkoxy” illustratively and preferably represents methoxy, ethoxy, n-propoxy, isopropoxy, tert-butoxy, n-pentoxy and n-hexoxy.

The term “alkanoyl” illustratively and preferably represents acetyl and propanoyl.

The term “alkylamino” represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propyl-amino, N-t-butyl-N-methylamino, N-ethyl-N-n-pentylamino and N-n-hexyl-N-methylamino.

The terms “alkylaminocarbonyl” or “alkylcarbamoyl” represent an alkylaminocarbonyl radical having one or two (independently selected) alkyl substituents, illustratively and preferably representing methylaminocarbonyl, ethylaminocarbonyl, n-propylaminocarbonyl, isopropylamino-carbonyl, tert-butylaminocarbonyl, n-pentylaminocarbonyl, n-hexylaminocarbonyl, N,N-dimethyl-aminocarbonyl, N,N-diethylaminocarbonyl, N-ethyl-N-methylaminocarbonyl, N-methyl-N-n-propylaminocarbonyl, N-isopropyl-N-n-propylaminocarbonyl, N-t-butyl-N-methylaminocarbonyl, N-ethyl-N-n-pentylamino-carbonyl and N-n-hexyl-N-methylaminocarbonyl.

The term “alkylaminosulphonyl” represents an alkylaminosulphonyl radical having one or two (independently selected) alkyl substituents, illustratively and preferably representing methylaminosulphonyl, ethylaminosulphonyl, n-propylaminosulphonyl, isopropylaminosulphonyl, tert-butylamino-sulphonyl, n-pentylaminosulphonyl, n-hexyl-aminosulphonyl, N,N-dimethylaminosulphonyl, N,N-diethylaminosulphonyl, N-ethyl-N-methylaminosulphonyl, N-methyl-N-n-propylaminosulphonyl, N-isopropyl-N-n-propylaminosulphonyl, N-t-butyl-N-methylaminosulphonyl, N-ethyl-N-n-pentyl-aminosulphonyl and N-n-hexyl-N-methylaminosulphonyl.

The term “alkylsulphonylamino” illustratively and preferably represents methylsulphonylamino, ethyl-sulphonylamino, n-propylsulphonylamino, isopropylsulphonylamino, tert-butyl-sulphonylamino, n-pentylsulphonylamino and n-hexylsulphonylamino.

The term “alkoxycarbonyl” illustratively and preferably represents methoxycarbonyl, ethoxycarbonyl, n-propoxycarbonyl, isopropoxycarbonyl, tert-butoxycarbonyl, n-pentoxycarbonyl and n-hexoxycarbonyl. Alkoxycarbonylamino illustratively and preferably represents methoxy-carbonylamino, ethoxycarbonylamino, n-propoxycarbonylamino, isopropoxycarbonylamino, tert-butoxycarbonylamino, n-pentoxycarbonylamino and n-hexoxycarbonylamino.

The term “alkanoylamino” illustratively and preferably represents acetylamino and ethylcarbonylamino.

The term “cycloalkyl” as used herein and for the cycloalkyl moieties in cycloalkylamino and in cycloalkylcarbonyl represents a cycloalkyl group having generally 3 to 8 and preferably 5 to 7 carbon atoms, illustratively and preferably representing cyclopropyl, cyclobutyl, cyclopentyl, cyclohexyl and cycloheptyl.

The term “cycloalkylamino” represents a cycloalkylamino radical having one or two (independently selected) cycloalkyl substituents, illustratively and preferably representing cyclopropylamino, cyclo-butylamino, cyclopentylamino, cyclohexylamino and cycloheptylamino.

The term “cycloalkylcarbonyl” illustratively and preferably represents cyclopropylcarbonyl, cyclobutylcarbonyl, cyclopentylcarbonyl, cyclohexylcarbonyl and cycloheptylcarbonyl.

The term “aryl” as used herein and for the aryl moieties in arylamino and in arylcarbonyl represents a mono-to tricyclic aromatic carbocyclic radical having generally 6 to 14 carbon atoms, illustratively and preferably representing phenyl, naphthyl and phenanthrenyl.

The term “arylamino” represents an arylamino radical having one or two (independently selected) aryl substituents, illustratively and preferably representing phenylamino, diphenylamino and naphthylamino.

The term “arylcarbonyl” illustratively and preferably represents phenylcarbonyl and naphthylcarbonyl.

The term “heteroaryl” as used herein and for the “heteroaryl moieties of heteroarylamino and heteroarylcarbonyl represents an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

The term “heteroarylamino” represents an heteroarylamino radical having one or two (independently selected) heteroaryl substituents, illustratively and preferably representing thienylamino, furylamino, pyrrolylamino, thiazolylamino, oxazolylamino, imidazolyl-amino, pyridylamino, pyrimidylamino, pyridazinylamino, indolylamino, indazolylamino, benzofuranylamino, benzothiophenylamino, quinolinylamino, isoquinolinylamino.

The term “heteroarylcarbonyl” illustratively and preferably represents thienylcarbonyl, furylcarbonyl, pyrrolylcarbonyl, thiazolylcarbonyl, oxazolylcarbonyl, imidazolylcarbonyl, pyridylcarbonyl, pyrimidylcarbonyl, pyridazinylcarbonyl, indolylcarbonyl, indazolylcarbonyl, benzofuranyl-carbonyl, benzothiophenylcarbonyl, quinolinylcarbonyl, isoquinolinylcarbonyl.

The term “heterocyclyl” as used herein and for the heterocyclyl moieties of heterocyclylcarbonyl represents a mono- or polycyclic, preferably mono- or bicyclic, nonaromatic heterocyclic radical having generally 4 to 10 and preferably 5 to 8 ring atoms and up to 3 and preferably up to 2 hetero atoms and/or hetero groups selected from the group consisting of N, O, S, SO and SO₂. The heterocyclyl radicals can be saturated or partially unsaturated. Preference is given to 5- to 8-membered monocyclic saturated heterocyclyl radicals having up to two hetero atoms selected from the group consisting of O, N and S, such as illustratively and preferably tetrahydrofuran-2-yl, pyrrolidin-2-yl, pyrrolidin-3-yl, pyrrolinyl, piperidinyl, morpholinyl, perhydroazepinyl.

The term “heterocyclylcarbonyl” illustratively and preferably represents tetrahydrofuran-2-carbonyl, pyrroli-dine-2-carbonyl, pyrrolidine-3-carbonyl, pyrrolinecarbonyl, piperidinecarbonyl, morpholine-carbonyl, perhydroazepinecarbonyl.

The specific and preferred definitions given for the individual radicals or moieties R¹, R², R³, R⁴, R⁵ and R⁶ herein below are valuable on their own as well as in combination. As will be understood preferred are compounds of formula (I) wherein one ore more of the individual radicals and moieties R¹, R², R³, R⁴, R⁵ and R⁶ have one of the meanings indicated as preferred herein-below and wherein the remaining radicals and moities are as specified hereinbefore. Most preferred are compounds of formula (I) wherein all of the individual radicals and moieties W R¹, R², R³, R⁴, R⁵ and R⁶ have one of the meanings indicated as preferred herein-below.

Preferred are compounds of formula (I), wherein the individual moieties have one of the preferred meanings given in the specification.

Preferred are compounds of formula (I) according to the invention, wherein

R¹ represents

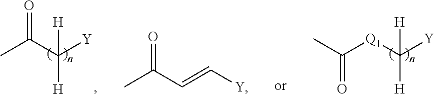

in which
n represents an integer of 0 to 2;
Q₁ represents —NH—, —N(C₁₋₆ alkyl)-, or —O—;
Y represents C₁₋₆ alkyl, C₃₋₈ cycloalkyl optionally substituted by C₁₋₆ alkyl, C₃₋₈ cycloalkyl fused by benzene selected from the group consisting of indenyl, and tetrahydronaphthyl, aryl selected from the group consisting of phenyl and naphthyl, or heteroaryl selected from the group consisting of indolyl, quinolyl, benzofuranyl, furanyl, chromanyl, and pyridyl, wherein said aryl and heteroaryl are optionally substituted at a substitutable position with one or more substituents selected from the group consisting of cyano, halogen, nitro, pyrrolyl, sulfamoyl, C₁₋₆ alylaminosulfonyl, di(C₁₋₆ alkyl)aminosulfonyl, phenyloxy, phenyl, C₁₋₆ alkylamino, di(C₁₋₆)alkylamino, C₁₋₆ alkoxycarbonyl, C₁₋₆ alkanoylamino, carbamoyl, C₁₋₆ alkylcarbamoyl, di-(C₁₋₆ alkyl) carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen; and
   R² represents hydrogen.

Preferred as well are compounds of formula (I) according to the invention, wherein R³ represents C₁₋₆ alkoxy optionally mono-, di-, or tri-substituted by halogen,

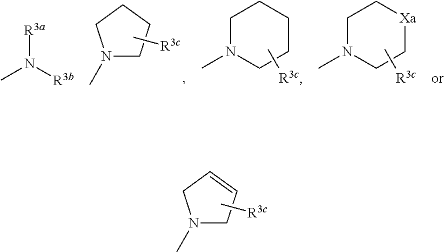

in which
R^{3a} and R^{3b} independently represent C₁₋₆ alkyl optionally substituted by hydroxy, carboxy, C₃₋₈, cycloalkyl, carbamoyl, C₁₋₆ alkylcarbamoyl, di(C₁₋₆ alkyl)carbamoyl, C₃₋₈, cycloalkylcarbamoyl, C₃₋₈ heterocyclocarbonyl, C₁₋₆ alkylamino, di(C₁₋₆) alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl) carbamoyl; and
Xa represents-O—, —S— or —N(R^{3d})—, in which R^{3d} represents C₁₋₆ alkyl.

Particularly preferred are compounds of formula (I), selected from compounds of formula (I-i) or pharmaceutically acceptable salts thereof,

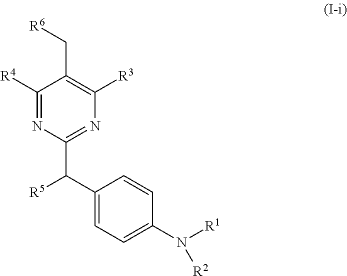

wherein
R¹ represents

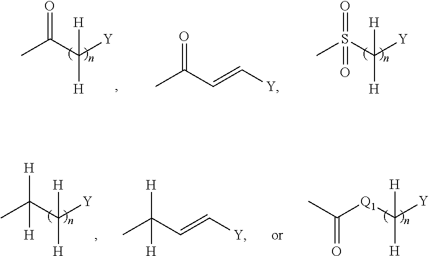

wherein
n represents an integer of 0 to 2;
Q₁ represents —NH—, —N(C₁₋₆ alkyl)-, or —O—;
Y represents phenyl, naphthyl, indolyl, quinolyl, benzofuranyl, furanyl or pyridyl, wherein said phenyl, naphthyl, indolyl, quinolyl, benzofuranyl, furanyl and pyridyl are optionally substituted at a substitutable position with one or two substituents selected from the group consisting of cyano, halogen, nitro, phenyloxy, phenyl, C₁₋₆ alkyl optionally mono-, di-, or tri-substituted by halogen, C₁₋₆ alkoxy optionally mono-, di-, or tri-substituted by halogen and C₁₋₆ alkylthio optionally mono-, di-, or tri-substituted by halogen;
   R² represents hydrogen or C₁₋₆ alkyl;
   R³ represents

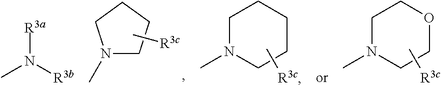

in which
R^{3a} and R^{3b} independently represent C₃₋₈, cycloalkyl, or C₁₋₆ alkyl optionally substituted by C₃₋₈, cycloalkyl, carbamoyl, C₁₋₆ alkylcarbamoyl, phenyl-substituted C₁₋₆ alkylcarbamoyl, C₁₋₆ alkylcarbamoyl, di (C₁₋₆ alkyl) carbamoyl, C₃₋₆ cycloalkylcarbamoyl, C₃₋₈ heterocyclocarbonyl, C₁₋₆ alkylamino, di(C₁₋₆) alkylamino or C₁₋₆ alkoxy,
R^{3c} represents hydrogen, hydroxy, carboxy, or C₁₋₆ alkyl optionally substituted by hydroxy, carboxy or (phenyl-substituted C₁₋₆ alkyl)carbamoyl;
   R⁴ represents hydrogen, chloro, bromo, C₁₋₆ alkoxy, di (C₁₋₆ alkyl) amino or C₁₋₆ alkyl optionally substituted by C₁₋₆ alkoxy;
   R⁵ represents hydrogen, or methyl; and
   R⁶ represents carboxy or tetrazolyl.

One specific embodiment of the invention relates to compounds of formula (I) and pharmaceutically acceptable salts thereof, wherein the compound of formula (I) is

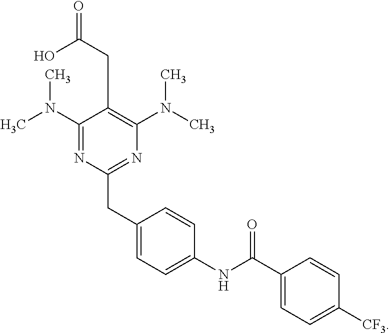 [SMILES: CN(C)c1nc(Cc2ccc(NC(=O)c3ccc(C(F)(F)F)cc3)cc2)nc(N(C)C)c1CC(=O)O]

A further embodiment of the present invention relates to compounds of formula (I), wherein the compounds of formula (I) are present in the form of the individual optical isomers, mixtures of the individual enantiomers or racemates, preferably in the form of the enantiomerically pure compounds.

A further embodiment of the present invention relates to compounds of formula (I), wherein the compounds of formula (I) are present in the form of the acid addition salts thereof with pharmacologically acceptable acids as well as optionally in the form of the solvates and/or hydrates.

In a particular embodiment of the invention the hair loss to be treated or prevented is related to androgenic alopecia, in particular to male pattern baldness or to female pattern baldness.

Another aspect of the present invention relates to the compounds of formula (I) or pharmaceutically acceptable salts thereof for use for stimulating hair growth, in particular for stimulating hair growth in human.

The at least one compound of formula (I) or the pharmaceutically acceptable salt thereof is preferably administered systemically or topically.

### Preparation

The compounds according to the invention may be obtained using methods of synthesis which are known to a person skilled in the art and described in the literature of organic synthesis. Preferably the compounds are obtained in analogy to the methods of preparation explained in more detail in WO 2004/096777 A1.

### Indications/Use

The present invention further relates to the use of at least one compound of formula (I) or a pharmaceutically acceptable salt thereof as defined herein for the manufacture of a medicament useful for the treatment or prevention of hairloss.

The at least one compound of formula (I) or the pharmaceutically acceptable salt thereof is preferably being used for the treatment or prevention of hairloss related to androgenic alopecia, in particular to male pattern baldness or to female pattern baldness.

According to the present invention the at least one compound of formula (I) or the pharmaceutically acceptable salt thereof is preferably used systemically or topically.

Another embodiment of the present invention relates to the manufacturing of a medicament for stimulating hair growth, in particular hair growth in a human. The at least one compound of formula (I) or the pharmaceutically acceptable salt thereof is preferably used systemically or topically.

### Method of Treatment

The compounds of formula (I) or the pharmaceutically acceptable salt thereof according to the present invention are useful in the prevention and/or treatment of hair loss.

Accordingly the present invention further relates to a method of treating or preventing hairloss, in particular hairloss in a human, said method comprising the step of administering a therapeutically effective amount of at least one compound of formula (I) or a pharmaceutically acceptable salt thereof as defined herein.

In a particular embodiment of the present invention the hair loss to be treated or prevented is related to androgenic alopecia, in particular to male pattern baldness or female pattern baldness.

Preferably the at least one compound of formula (I) or the pharmaceutically acceptable salt thereof is administered systemically or topically

The compounds of formula (I) according to the present invention are also useful for stimulating hair growth.

Accordingly the present invention further relates to a method of stimulating hair growth, in particular hair growth in a human, said method comprising the step of administering a therapeutically effective amount of at least one compound of formula (I) or a pharmaceutically acceptable salt thereof as defined herein.

Preferably the at least one compound of formula (I) or the pharmaceutically acceptable salt thereof is administered systemically or topically.

### Pharmaceutical Forms

Another aspect of the present invention relates to pharmaceutical compositions for preventing or treating hairloss or for stimulating hairgrowth which are characterized in that they contain a compound of formula (I) or a pharmaceutically acceptable salt thereof as defined herein.

Accordingly the present invention relates to a pharmaceutical composition for use in the prevention or treatment of hairloss, in particular hair loss in humans, containing at least one compound of formula (I) or a pharmaceutically acceptable salt thereof as defined herein.

Another embodiment of the present invention relates to pharmaceutical composition for use in method of stimulating hair growth, in particular hair growth in a human, said method comprising the step of administering a therapeutically effective amount of at least one compound of formula (I) or a pharmaceutically acceptable salt thereof as defined herein.

The pharmaceutical compositions of the present invention may preferably be administered systemically or topically. Accordingly the present invention further relates to suitable topical or systemical pharmaceutical formulations for use in the novel methods of treatment and uses of the present invention.

The dosage of the compounds according to the invention is naturally highly dependent on the method of administration.

The term “therapeutically effective amount” as used herein refers to a daily dosage of the compounds of formula (I) or the pharmaceutically acceptable salts thereof in a range from 0.5 to 1000 mg, preferably 1 to 500 mg, more preferably from 5 to 200 mg.

The content of the pharmaceutically active compound(s) should be in the range from 0.05 to 90 wt.-%, preferably 0.1 to 50 wt.-% of the total weight of the pharmaceutical composition.

The pharmaceutical compositions containing the compound or composition may be administered to a subject by any method known to a person skilled in the art, e.g. parenterally, transdermally, intravenously, intradermally or subcutaneously.

The pharmaceutical compositions containing the compounds of the present invention can be administered in a wide variety of therapeutic dosage forms in conventional vehicles for systemic administration, as for example, by oral administration in the form of tablets, capsules, solutions, suspensions, suppositories, powders or by intravenous injection.

One particular embodiment of the present invention relates to pharmaceutical composition as defined herein, wherein the pharmaceutical composition is a tablet, capsule, pill, solution, suspension, dispersion, emulsion or suppository for systemical administration.

In another embodiment, the pharmaceutical composition is administered topically to the body surfaces and is thus formulated in a form suitable for topical administration.

Suitable topical formulations include gels, ointments, creams, lotions, drops and the like.

Another particular embodiment of the present invention therefor relates to the pharmaceutical composition as defined herein, wherein the pharmaceutical composition is a solution, suspension, emulsion, dispersion, cream, ointment, gel, lotion, shampoo or aerosol for topical administration.

Solutions are prepared in the usual way, e.g. with the addition of isotonic agents, preservatives such as p-hydroxybenzoates or stabilizers such as alkali metal salts of ethylenediaminetetraacetic acid, optionally using emulsifiers and/or dispersants, while if water is used as diluent, for example, organic solvents may optionally be used as solubilisers or dissolving aids, and the solutions may be transferred into injection vials or ampoules or infusion bottles.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number or layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavor enhancer, e.g. a flavoring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include but are not limited to water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

For oral use the tablets may obviously contain, in addition to the carriers specified, additives such as sodium citrate, calcium carbonate and dicalcium phosphate together with various additional substances such as starch, preferably potato starch, gelatine and the like. Lubricants such as magnesium stearate, sodium laurylsulphate and talc may also be used to produce the tablets. In the case of aqueous suspensions the active substances may be combined with various flavor enhancers or colorings in addition to the above-mentioned excipients.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

The pharmaceutical compositions provided herein may be formulated as controlled-release compositions or as immediate-release composition.

Compounds of the formula (I) of the present invention can be, but are not limited to be, prepared by the Method [A] to [J] as described in WO 2004/096777.

The following examples serve to further illustrate the present invention without restricting its scope.

### EXAMPLES

The effect of the present compounds was examined by the following assays and pharmacological tests.

### Biological Assay

### [Preparation of Human CRTH2-Transfected L1. 2 Cell Line]

Human CRTH2 cDNA was amplified from human eosinophil cDNA with gene specific primers containing restriction sites for cloning into pEAK vector (Edge Bio Systems). The human CRTH2 cDNA was cloned into the mammalian expression vector pEAK. This expression plasmid (40 pg) was transfected into L1. 2 cells, at a cell density of 1×107 cells/500 p1, by using electroporation apparatus (Gene Pulser II, BioRad) at 250V/1,000 pF. One day after the transfection, puromycin (1 ug/mi, Sigma) was added into the cell culture plates. Two weeks after the transfection, grown cells were picked up for further growth.

### [Measurement of Ca²⁺ Mobilization in the Human CRTH2-Transfected L1. 2 Cell Line]

Ca²⁺ loading buffer was prepared by mixing 5 zu of Fluo-3AM (2 mM in DMSO, final 1 uM, Molecular Probes) and 10 u1 of pluronic F-127 (Molecular Probes) and diluting the resulting mixture in 10 ml of Ca²⁺ assay buffer (20 mM HEPES pH 7.6, 0.1% BSA, 1 mM probenecid, Hanks' solution). The CRTH2 transfected cells which were prepared in Example 1 were washed with PBS, resuspended in Ca2+ loading buffer at 1×10′cells/ml, and incubated for 60 min at room temperature. After incubation, cells were washed and resuspended in Ca²⁺ assay buffer, then dispensed into transparent-bottom 96-well plates (&num; 3631, Costar) at 2×105 cells/well. Cells were incubated with various concentrations of test compound for 5 minutes at room temperature. The emitted 480 nm fluorescence was measured on FDSS6000, a Ca²⁺-measurement apparatus (Hamamatsu Photonics, Hamamatsu, Japan). The transfectant showed PGD2-induced Ca²⁺ mobilization in a concentration-dependent manner.

Assay results are shown in table 1 below. The data corresponds to the compounds as yielded by solid phase synthesis and thus to levels of purity of about 40 to 90%. For practical reasons, the compounds are grouped in four classes of activity as follows: IC₅₀=A (< or =) 10 nM<B (< or =) 100 nM<C (< or =) 500 nM<D. The compounds of the present invention also show excellent selectivity, and potent activity in Assays 2,3 and 4 described above.

TABLE 1

| Example | Structure | Activity class |
|---|---|---|
| 1 | 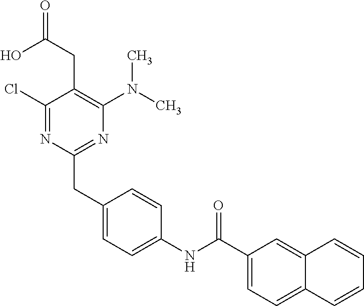 [SMILES: CN(C)c1nc(Cc2ccc(NC(=O)c3ccc4ccccc4c3)cc2)nc(Cl)c1CC(=O)O] | A |
| 2 | 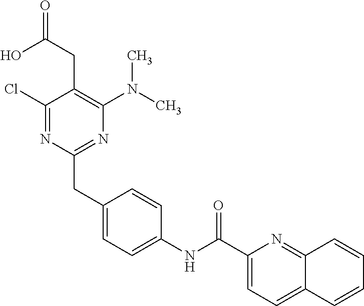 [SMILES: CN(C)c1nc(Cc2ccc(NC(=O)c3ccc4ccccc4n3)cc2)nc(Cl)c1CC(=O)O] | A |
| 3 | 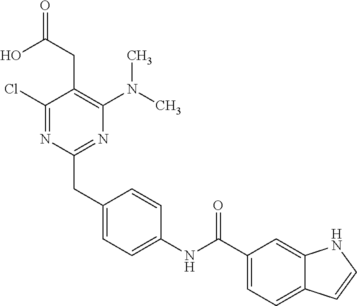 [SMILES: CN(C)c1nc(Cc2ccc(NC(=O)c3ccc4cc[nH]c4c3)cc2)nc(Cl)c1CC(=O)O] | A |
| 4 | 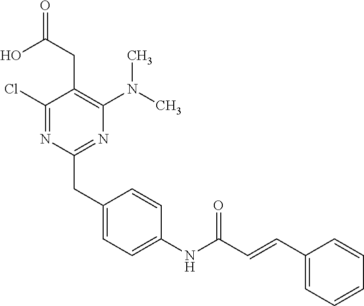 [SMILES: CN(C)c1nc(Cc2ccc(NC(=O)/C=C/c3ccccc3)cc2)nc(Cl)c1CC(=O)O] | A |
| 5 | 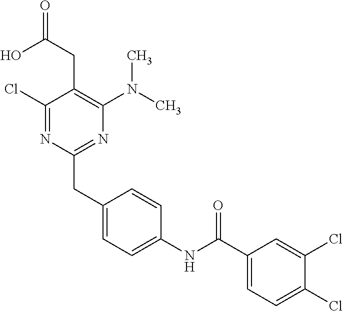 [SMILES: CN(C)c1nc(Cc2ccc(NC(=O)c3ccc(Cl)c(Cl)c3)cc2)nc(Cl)c1CC(=O)O] | A |
| 6 | 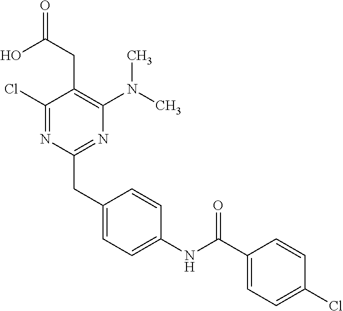 [SMILES: CN(C)c1nc(Cc2ccc(NC(=O)c3ccc(Cl)cc3)cc2)nc(Cl)c1CC(=O)O] | A |
| 7 | 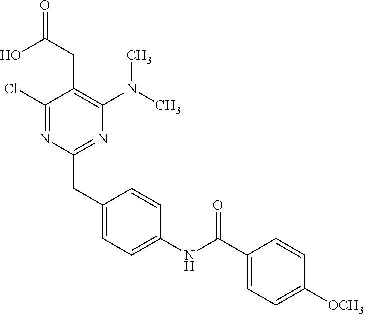 [SMILES: COc1ccc(C(=O)Nc2ccc(Cc3nc(Cl)c(CC(=O)O)c(N(C)C)n3)cc2)cc1] | A |
| 8 | 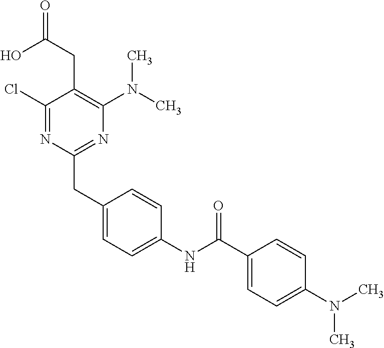 [SMILES: CN(C)c1ccc(C(=O)Nc2ccc(Cc3nc(Cl)c(CC(=O)O)c(N(C)C)n3)cc2)cc1] | A |
| 9 | 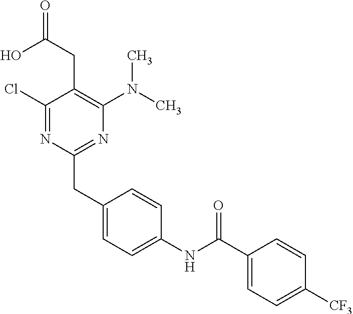 [SMILES: CN(C)c1nc(Cc2ccc(NC(=O)c3ccc(C(F)(F)F)cc3)cc2)nc(Cl)c1CC(=O)O] | A |
| 10 | 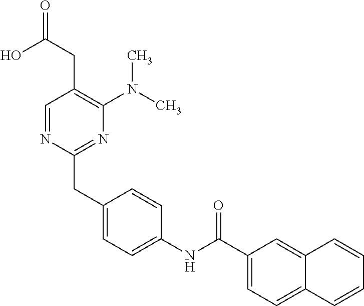 [SMILES: CN(C)c1nc(Cc2ccc(NC(=O)c3ccc4ccccc4c3)cc2)ncc1CC(=O)O] | A |
| 11 | 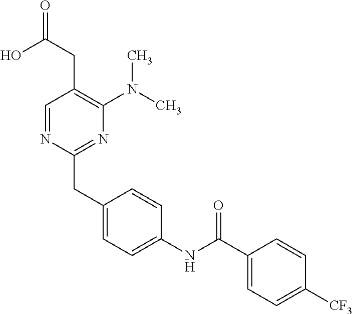 [SMILES: CN(C)c1nc(Cc2ccc(NC(=O)c3ccc(C(F)(F)F)cc3)cc2)ncc1CC(=O)O] | A |
| 12 | 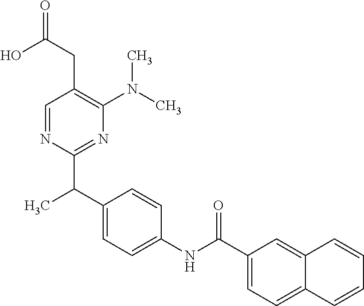 [SMILES: CC(c1ccc(NC(=O)c2ccc3ccccc3c2)cc1)c1ncc(CC(=O)O)c(N(C)C)n1] | A |
| 13 | 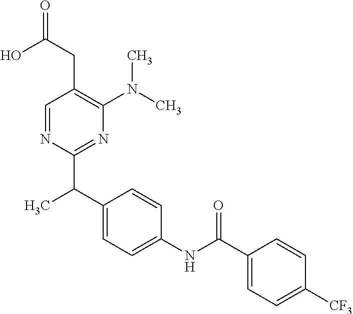 [SMILES: CC(c1ccc(NC(=O)c2ccc(C(F)(F)F)cc2)cc1)c1ncc(CC(=O)O)c(N(C)C)n1] | A |
| 14 | 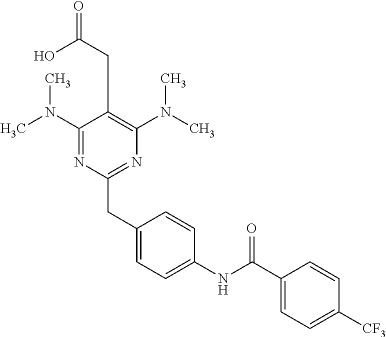 [SMILES: CN(C)c1nc(Cc2ccc(NC(=O)c3ccc(C(F)(F)F)cc3)cc2)nc(N(C)C)c1CC(=O)O] | A |
| 15 | 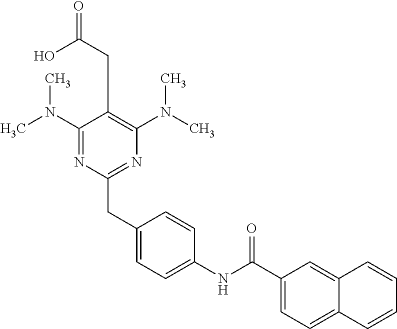 [SMILES: CN(C)c1nc(Cc2ccc(NC(=O)c3ccc4ccccc4c3)cc2)nc(N(C)C)c1CC(=O)O] | B |
| 16 | 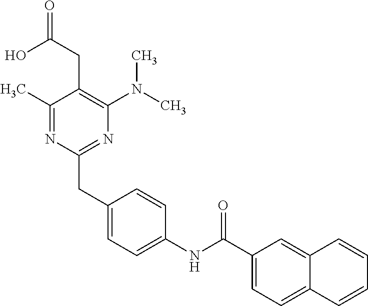 [SMILES: Cc1nc(Cc2ccc(NC(=O)c3ccc4ccccc4c3)cc2)nc(N(C)C)c1CC(=O)O] | A |
| 17 | 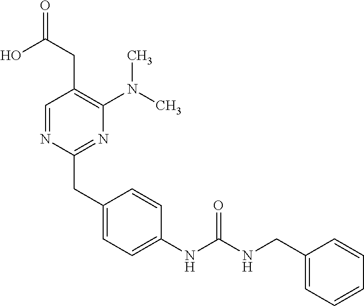 [SMILES: CN(C)c1nc(Cc2ccc(NC(=O)NCc3ccccc3)cc2)ncc1CC(=O)O] | B |
| 18 | 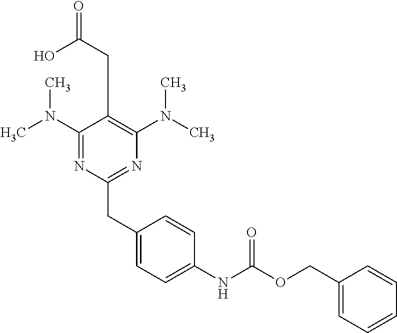 [SMILES: CN(C)c1nc(Cc2ccc(NC(=O)OCc3ccccc3)cc2)nc(N(C)C)c1CC(=O)O] | A |
| 19 | 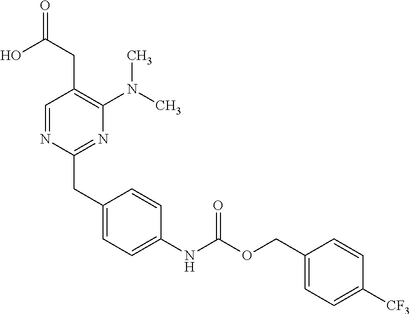 [SMILES: CN(C)c1nc(Cc2ccc(NC(=O)OCc3ccc(C(F)(F)F)cc3)cc2)ncc1CC(=O)O] | A |
| 20 | 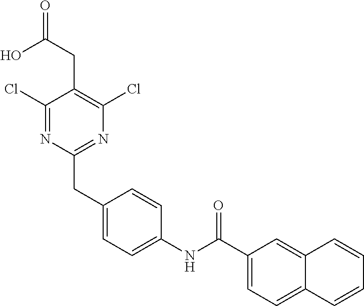 [SMILES: O=C(O)Cc1c(Cl)nc(Cc2ccc(NC(=O)c3ccc4ccccc4c3)cc2)nc1Cl] | A |
| 21 | 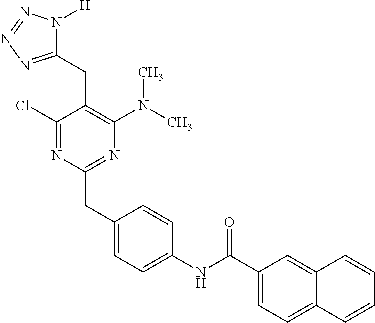 [SMILES: CN(C)c1nc(Cc2ccc(NC(=O)c3ccc4ccccc4c3)cc2)nc(Cl)c1Cc1nnn[nH]1] | A |
| 22 | 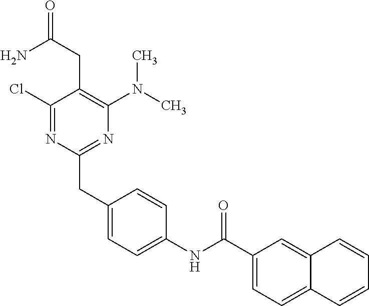 [SMILES: CN(C)c1nc(Cc2ccc(NC(=O)c3ccc4ccccc4c3)cc2)nc(Cl)c1CC(N)=O] | B |

1. A method of treating or preventing hairloss in a patient in need thereof, the method comprising administering to the patient an effective amount of a compound of formula (I) or a pharmaceutically acceptable salt thereof 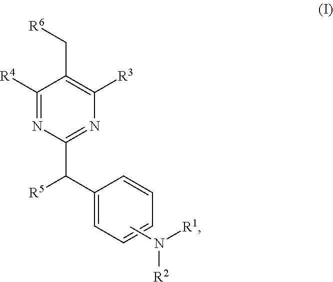
wherein:
R¹ is hydrogen, 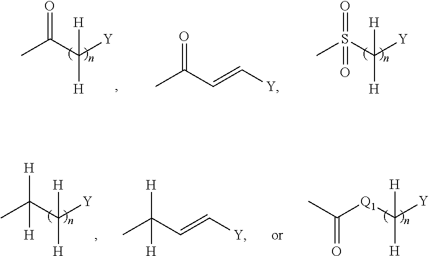
wherein
n is 0 to 6,
Q₁ is —NH—, —N(C₁₋₆alkyl)-, or —O—, and
Y is hydrogen, C₁₋₆ alkyl, C₃₋₈ cycloalkyl optionally substituted by C₁₋₆ alkyl, C₃₋₈ cycloalkyl fused by benzene, aryl, or heteroaryl, wherein the aryl and heteroaryl are optionally substituted at a substitutable position with one or more substituents selected from the group consisting of cyano, halogen, nitro, guanidino, pyrrolyl, sulfamoyl, C₁₋₆ alkylaminosulfonyl, di (C₁₋₆ alkyl) aminosulfonyl, phenyloxy, phenyl, amino, C₁₋₆ alkylamino, di(C₁₋₆)alkylamino, C₁₋₆ alkoxycarbonyl, C₁₋₆ alkanoyl, C₁₋₆ alkanoylamino, carbamoyl, C₁₋₆ alkylcarbamoyl, di(C₁₋₆ alkyl)carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen, and C₁₋₆ alkylthio optionally mono-, di-, or tri-substituted by halogen, or aryl fused by 1,3-dioxolane;
R² is hydrogen or C₁₋₆ alkyl;
R³ is halogen, C₁₋₆ alkoxy optionally mono-, di-, or tri-substituted by halogen, 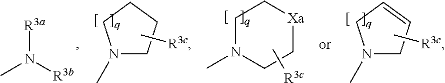
wherein
R^{3a} and R^{3b} are each independently C₃₋₈ cycloalkyl, or C₁₋₆ alkyl optionally substituted by hydroxy, carboxy, C₃₋₈ cycloalkyl, carbamoyl, C₁₋₆ alkylcarbamoyl, aryl-substituted C₁₋₆ alkylcarbamoyl, C₁₋₆ alkylcarbamoyl, di (C₁₋₆ alkyl) carbamoyl, C₃₋₈ cycloalkylcarbamoyl, C₃₋₈ heterocyclocarbonyl, (C₁₋₆)alkylamino, di (C₁₋₆) alkylamino, or C₁₋₆ alkoxy,
q is 1 to 3,
R^{3c} is hydrogen, hydroxy, carboxy, or C₁₋₆ alkyl optionally substituted by hydroxy, carboxy, or (phenyl-substituted C₁₋₆ alkyl) carbamoyl, and
Xa is —O—, —S—, or —N(C₁₋₆ alkyl);
R⁴ is hydrogen, halogen, C₁₋₆ alkoxy, di(C₁₋₆ alkyl)amino, or C₁₋₆ alkyl optionally substituted by C₁₋₆ alkoxy, or mono-, di-, or tri-substituted by halogen;
R⁵ is hydrogen; or C₁₋₆ alkyl; and
R⁶ is carboxy, carboxamide, nitrile, or tetrazolyl.

2. The method according to claim 1, wherein:
R¹ is 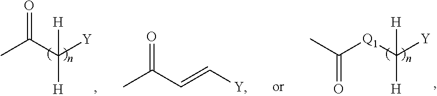
wherein
n is 0 to 2,
Q₁ is —NH—, —N(C₁₋₆ alkyl)-, or —O—, and
Y is C₁₋₆ alkyl, C₃₋₈ cycloalkyl optionally substituted by C₁₋₆ alkyl, indenyl, tetrahydronaphthyl, phenyl, naphthyl, indolyl, quinolyl, benzofuranyl, furanyl, chromanyl, or pyridyl, wherein the aryl and heteroaryl are optionally substituted at a substitutable position with one or more substituents selected from the group consisting of cyano, halogen, nitro, pyrrolyl, sulfamoyl, C₁₋₆ alkylaminosulfonyl, di(C₁₋₆ alkyl)aminosulfonyl, phenyloxy, phenyl, C₁₋₆ alkylamino, di(C₁₋₆)alkylamino, C₁₋₆ alkoxycarbonyl, C₁₋₆ alkanoylamino, carbamoyl, C₁₋₆ alkylcarbamoyl, di-(C₁₋₆ alkyl) carbamoyl, C₁₋₆ alkylsulfonyl, C₁₋₆ alkyl optionally mono-, di-, or tri-substituted by halogen, C₁₋₆ alkoxy optionally mono-, di-, or tri-substituted by halogen, and C₁₋₆ alkylthio optionally mono-, di-, or tri-substituted by halogen; and
R² is hydrogen.

3. The method according to claim 1, wherein:
R³ is C₁₋₆ alkoxy optionally mono-, di-, or tri-substituted by halogen, 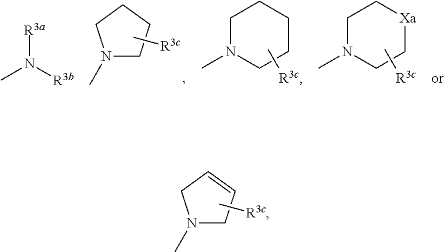
wherein
R^{3a} and R^{3b} are each independently C₁₋₆ alkyl optionally substituted by hydroxy, carboxy, C₃₋₈ cycloalkyl, carbamoyl, C₁₋₆ alkylcarbamoyl, di(C₁₋₆ alkyl)carbamoyl, C₃₋₈ cycloalkylcarbamoyl, C₃₋₈ heterocyclocarbonyl, C₁₋₆ alkylamino, di(C₁₋₆) alkylamino, or C₁₋₆ alkoxy,
R^{3c} is hydrogen, hydroxy, carboxy, or C₁₋₆ alkyl optionally substituted by hydroxy, carboxy, or (phenyl-substituted C₁₋₆ alkyl) carbamoyl; and
Xa is —O—, —S—, or —N(C₁₋₆ alkyl).

4. The method according to claim 1, wherein the compound of formula (I) or a pharmaceutically acceptable salt thereof is selected from a compound of formula (I-i) 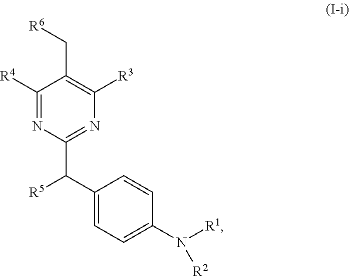
wherein:
R¹ is 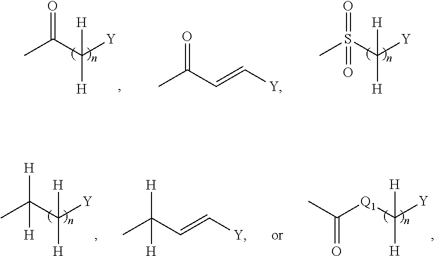
wherein
n is 0 to 2;
Q₁ is —NH—, —N(C₁₋₆ alkyl)-, or —O—;
Y is phenyl, naphthyl, indolyl, quinolyl, benzofuranyl, furanyl, or pyridyl, wherein the phenyl, naphthyl, indolyl, quinolyl, benzofuranyl, furanyl, and pyridyl are optionally substituted at a substitutable position with one or two substituents selected from the group consisting of cyano, halogen, nitro, phenyloxy, phenyl, C₁₋₆ alkyl optionally mono-, di-, or tri-substituted by halogen, C₁₋₆ alkoxy optionally mono-, di-, or tri-substituted by halogen, and C₁₋₆ alkylthio optionally mono-, di-, or tri-substituted by halogen;
R² is hydrogen or C₁₋₆ alkyl;
R³ is 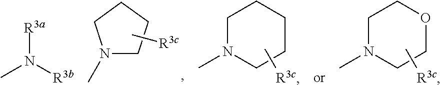
wherein
R^{3a} and R^{3b} are each independently C₃₋₈ cycloalkyl, or C₁₋₆ alkyl optionally substituted by C₃₋₈ cycloalkyl, carbamoyl, C₁₋₆ alkylcarbamoyl, phenyl-substituted C₁₋₆ alkylcarbamoyl, C₁₋₆ alkylcarbamoyl, di (C₁₋₆ alkyl) carbamoyl, C₃₋₆ cycloalkylcarbamoyl, C₃₋₈ heterocyclocarbonyl, C₁₋₆ alkylamino, di(C₁₋₆) alkylamino, or C₁₋₆ alkoxy, and R^{3c} is hydrogen, hydroxy, carboxy, or C₁₋₆ alkyl optionally substituted by hydroxy, carboxy, or (phenyl-substituted C₁₋₆ alkyl)carbamoyl;
R⁴ is hydrogen, chloro, bromo, C₁₋₆ alkoxy, di (C₁₋₆ alkyl) amino, or C₁₋₆ alkyl optionally substituted by C₁₋₆ alkoxy;
R⁵ is hydrogen or methyl; and
R⁶ is carboxy or tetrazolyl.

5. The method according to claim 1, wherein the compound of formula (I) or a pharmaceutically acceptable salt thereof is

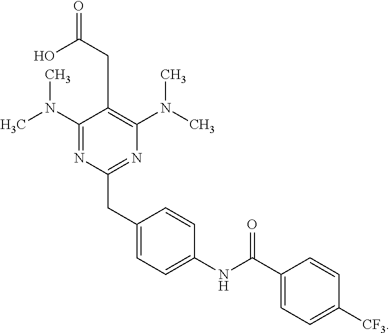 [SMILES: CN(C)c1nc(Cc2ccc(NC(=O)c3ccc(C(F)(F)F)cc3)cc2)nc(N(C)C)c1CC(=O)O]

6. The method according to claim 1, wherein the compound of formula (I) or a pharmaceutically acceptable salt thereof is administered in the form of a pharmaceutical composition a compound of formula (I) or a pharmaceutically acceptable salt thereof and a pharmaceutical excipient.

7. The method according to claim 6, wherein the pharmaceutical composition is a tablet, capsule, pill, solution, suspension, dispersion, emulsion, or suppository.

8. The method according to claim 6, wherein the pharmaceutical composition is a solution, suspension, emulsion, dispersion, cream, ointment, gel, lotion, shampoo, or aerosol.

9. The method according to claim 1, wherein the hairloss is related to androgenic alopecia.

10. The method according to claim 8, wherein the hairloss is related to androgenic alopecia.

11. The method according to claim 9, wherein the androgenic alopecia is male pattern baldness or female pattern baldness.

12.-15. (canceled)
